(19) United States
(12) Patent Application Publication
(10) Pub. No.: US 2022/0362772 A1
(43) Pub. Date: Nov. 17, 2022

(54) IMMOBILIZATION AND QUANTITATIVE MEASUREMENT OF DROPLETS

(71) Applicant: THE JOHNS HOPKINS UNIVERSITY, Baltimore, MD (US)

(72) Inventors: Tza-Huei Jeff WANG, Baltimore, MD (US); Christine M. O'KEEFE, Baltimore, MD (US); Aniruddha Mrithinjay KAUSHIK, Baltimore, MD (US)

(21) Appl. No.: 17/624,440

(22) PCT Filed: Jul. 1, 2020
(86) PCT No.: PCT/US2020/040554

§ 371 Date: Jan. 3, 2022

Related U.S. Application Data
(60) Provisional application No. 62/870,258, filed on Jul. 3, 2019.

(51) Int. Cl. B01L 3/00; C12Q 1/6858

(52) U.S. Cl. CPC B01L 3/502761; C12Q 1/6858; B01L 2200/0673; B01L 2300/0654; B01L 2300/18; C12Q 2600/154

(57) ABSTRACT

Provided herein are microfluidic devices for analyzing samples. In one aspect, the microfluidic device includes a body structure having a droplet compression chamber, a sieve structure in fluid communication with the droplet compression chamber, which sieve structure comprises an array of protrusions that extend from at least one surface of the body structure and define at least a portion of one or more fluidic circuits, and a port at least partially disposed in the body structure. Other aspects include kits, methods, systems, computer readable media, and related aspects for analyzing samples.

### RELATED APPLICATION

This application claims the benefit of, and priority to, U.S. Provisional Patent Application No. 62/870,258, filed Jul. 3, 2019, which is hereby incorporated by reference in its entirety.

### STATEMENT OF GOVERNMENT SUPPORT

This invention was made using U.S. Government support under NIH Grant Nos. R01AI117032, U01CA214165, and R01AI137272. The U.S. Government has certain rights in this invention.

### BACKGROUND

The ability to compartmentalize samples into droplets for high-throughput measurements has greatly improved sensitivity towards detection of rare molecules and has opened doors for new insights into single cell and single molecule analyses¹⁻⁶. Droplet platforms have been used to detect and analyze nucleic acid and protein biomarkers as well as single cells with drastically higher sensitivities than bulk methods1,⁷⁻¹¹. Furthermore, with advances in droplet microfluidic technologies, commercial platforms like BioRad QX200 ddPCR and RainDance RainDrop have become turnkey tools for a wide range of clinical applications, such as copy number variation¹²⁻¹⁴, biomarker detection¹⁵⁻¹⁸, gene expression analysis¹⁹⁻²¹, and pathogen detection^{22,23}.

Despite their tremendous advantages in sensitivity and throughput, most existing droplet platforms rely on sequential measurements of each droplet and its assay reactants at a single predetermined time point (“endpoint analysis”)²⁴⁻²⁷. This limitation precludes their utility in biological assays that rely on or benefit from time-resolved measurements of reactants (“real-time analysis”), such as molecular profiling using melting curve analysis²⁸⁻³⁴, cell growth monitoring^{35,36}, and enzyme kinetics observations³⁷⁻⁴⁰.

For effective real-time analysis of individual droplets, it is advantageous to immobilize the droplets such that they can be easily monitored over the course of the assay. However, previous attempts at developing such a real-time droplet analysis platform have resulted in a tradeoff, in which there is a reduction in either throughput or droplet capture efficiency. To ensure trapping of many or most of the droplets in a reaction, some methods directly generate droplets into a chamber or reservoir for reaction and analysis⁴¹⁻⁴⁵. However, once loaded, these devices are sealed and droplet generation halted while all reaction steps are performed in the chamber. Therefore, these “on-chip” methods are inherently limited in throughput, as the entire device is restricted to a single run of a single condition or sample, especially for multistep assays that contain time-consuming reaction or incubation steps. Even with multiple devices, the number of reactions or samples that may be parallelized is constrained by the two-dimensional surface area of commercial heaters. Moreover, the footprint of these chambers may be inefficiently utilized, as they retain the immiscible oil phase used in droplet generation.

In contrast, trapping strategies that are fundamentally compatible with “off-chip” droplet production allow high-throughput parallelization of time-limiting reaction steps on conventional 96- or 384-well plates before subsequently attempting to capture the droplets in an array/chip for analysis. These methods potentially enable simultaneous assessment of multiple reactions, a range of reaction conditions, or large patient/sample cohorts for time-limiting reaction steps in a high-throughput manner that is favorable for translational potential. In addition, the throughput of subsequent real-time analyses can be further enhanced by incorporating multiple trapping units on a single device. However, current “off-chip” trapping designs have poor capture efficiencies, only capturing and analyzing a small percentage of overall droplets^{35,39,46-48}, as a large number of droplets tend to bypass the storage chambers and are lost when loading the array.

Ultimately, loss in either throughput or droplet capture efficiency undermines the utility of current platforms towards clinical analyses of rare molecules or biomarkers and applications that utilize real-time monitoring to identify extremely rare variants, such as bacterial persistence⁴⁹, drug discovery⁵⁰, directed evolution⁵¹, antibiotic resistance⁵², and circulating biomarker analysis^{28,31-33,53}. Accordingly, there remains a need for systems, devices, methods, and related aspects of real-time droplet analysis.

### SUMMARY

This application discloses real-time droplet platforms and related aspects to perform high throughput, real-time simultaneous analyses of multiple droplets that improve space utilization, operational flexibility, and capture efficiency relative to pre-existing systems and techniques.

In one aspect, this disclosure provides a microfluidic device that includes a body structure having a droplet compression chamber that is structured to at least partially and simultaneously contain a plurality of droplets, at least one sieve structure in fluid communication with the droplet compression chamber, which sieve structure comprises an array of protrusions that extend from at least one surface of the body structure and define at least a portion of one or more fluidic circuits, and at least one port at least partially disposed in the body structure, which port is in fluid communication with the droplet compression chamber and/or the fluidic circuits. The droplet compression chamber and sieve structure compress and selectively immobilize the plurality of droplets and permit selective removal of carrier fluid from the droplet compression chamber through the fluidic circuits, when the plurality of droplets are disposed in the droplet chamber and positioned substantially above the fluidic circuits proximal to the array of protrusions. In some embodiments, the body structure comprises a first layer defining at least the portion of the droplet compression chamber that is structured to at least partially and simultaneously contain the plurality of droplets, and a second layer operably connected to the first layer, which second layer comprises the at least one sieve structure in fluid communication with the droplet compression chamber, wherein the at least one port is at least partially disposed in the first and/or second layer.

In some embodiments, the microfluidic device include one or more gaps (e.g., highways) within and/or proximal to the array of protrusions, which gaps substantially lack protrusions. In certain embodiments, the microfluidic device comprises the plurality of droplets. The plurality of droplets typically comprise a partitioned sample (e.g., a sample obtained from a subject). The plurality of droplets typically comprise a partitioned sample. In some embodiments, at least a portion of one or more of the protrusions comprise at least one cross-sectional shape selected from, for example, a square, a rectangle, an oval, a trapezoid, a circle, an irregular n-sided polygon, a regular n-sided polygon, and/or the like. In some embodiments, the droplet compression chamber is structured to at least partially and simultaneously contain the plurality of droplets at a density of at least about 110,000 droplets per square inch of at least one surface of the droplet compression chamber. In certain embodiments, the microfluidic device includes at least two ports at least partially disposed in the body structure and in fluid communication with the droplet compression chamber and/or the fluidic circuits, wherein at least a first port is configured to flow droplets into the droplet compression chamber and at least a second port is configured to flow carrier fluid out of the droplet compression chamber through the fluidic circuits. In some embodiments, a kit includes the microfluidic device.

In another aspect, the disclosure provides a method of analyzing a sample. The method includes receiving a mixture comprising a plurality of droplets and at least one carrier fluid in a droplet compression chamber of a microfluidic device through at least a first port of the microfluidic device that is in fluid communication with the droplet compression chamber, wherein the plurality of droplets comprises partitioned portions of the sample and wherein at least one sieve structure of the microfluidic device is in fluid communication with the droplet compression chamber, which sieve structure comprises an array of protrusions that extend from at least one surface of the microfluidic device and define at least a portion of one or more fluidic circuits. The method also includes removing at least a portion of the carrier fluid from the droplet compression chamber through at least a second port of the microfluidic device that is in fluid communication with the fluidic circuits to immobilize the plurality of droplets substantially above the fluidic circuits proximal to the array of protrusions to generate an immobilized population of droplets. In addition, the method also includes detecting at least one detectable signal from the immobilized population of droplets. In some embodiments, the plurality of droplets is in an aqueous phase and wherein the carrier fluid is in a non-aqueous phase. In certain embodiments, the detectable signal comprises a thermal and/or electromagnetic property of, or originating from, one or more members of the population of droplets or components thereof. In some embodiments, the sample comprises one or more cells and/or biomolecules.

In certain embodiments, the method includes detecting the detectable signal using a thermal and/or optical imaging device. In some embodiments, the method includes at least about 110,000 droplets per square inch of at least one surface of the droplet compression chamber. In some embodiments, the method also includes obtaining the sample (e.g., tissue, blood, urine, cerebrospinal fluid, etc.) from a subject. In certain embodiments, the method includes generating the plurality of droplets using at least one droplet generating device.

In another aspect, the disclosure provides a microfluidic system that includes a microfluidic device, comprising a body structure having a droplet compression chamber that is structured to at least partially and simultaneously contain a plurality of droplets, at least one sieve structure in fluid communication with the droplet compression chamber, which sieve structure comprises an array of protrusions that extend from at least one surface of the body structure and define at least a portion of one or more fluidic circuits, and at least one port at least partially disposed in the body structure, which port is in fluid communication with the droplet compression chamber and/or the fluidic circuits. The droplet compression chamber and sieve structure compress and selectively immobilize the plurality of droplets and permit selective removal of carrier fluid from the droplet compression chamber through the fluidic circuits, when the plurality of droplets are disposed in the droplet chamber and positioned substantially above the fluidic circuits proximal to the array of protrusions. The microfluidic system also includes a detection device configured to obtain detectable signal from the plurality of droplets, when the plurality of droplets are disposed in the droplet chamber and positioned substantially above the fluidic circuits proximal to the array of protrusions. In addition, the microfluidic system also includes a control device operably connected to the detection device, which control device comprises, or is capable of accessing, computer readable media comprising non-transitory computer-executable instructions which, when executed by at least one electronic processor perform at least detecting at least one detectable signal from the plurality of droplets, when the plurality of droplets are disposed in the droplet chamber and positioned substantially above the fluidic circuits proximal to the array of protrusions.

In some embodiments, the microfluidic system also includes a droplet generating device operably connected to the control device, which control device further comprises, or is capable of further accessing, computer readable media comprising non-transitory computer-executable instructions which, when executed by the at least one electronic processor further perform generating the plurality of droplets using the droplet generating device. In certain embodiments, the microfluidic system also includes a droplet treatment device operably connected at least to the control device, the droplet generating device, and the microfluidic device, which control device further comprises, or is capable of further accessing, computer readable media comprising non-transitory computer-executable instructions which, when executed by the at least one electronic processor further perform conveying the plurality of droplets from the droplet generating device to the droplet treatment device, treating the plurality of droplets received from the droplet generating device to generate treated droplets using the droplet treatment device, and conveying the treated droplets from the droplet treatment device to the microfluidic device.

In another aspect, the disclosure provides a computer readable media comprising non-transitory computer-executable instructions which, when executed by at least one electronic processor perform, or cause an operably connected microfluidic system component to perform, at least receiving a mixture comprising a plurality of droplets and at least one carrier fluid in a droplet compression chamber of a microfluidic device through at least a first port of the microfluidic device that is in fluid communication with the droplet compression chamber, wherein the plurality of droplets comprises partitioned portions of the sample and wherein at least one sieve structure of the microfluidic device is in fluid communication with the droplet compression chamber, which sieve structure comprises an array of protrusions that extend from at least one surface of the microfluidic device and define at least a portion of one or more fluidic circuits, removing at least a portion of the carrier fluid from the droplet compression chamber through at least a second port of the microfluidic device that is in fluid communication with the fluidic circuits to immobilize the plurality of droplets substantially above the fluidic circuits proximal to the array of protrusions to generate an immobilized population of droplets, and detecting at least one detectable signal from the immobilized population of droplets.

### BRIEF DESCRIPTION OF THE DRAWINGS

The accompanying drawings, which are incorporated in and constitute a part of this specification, illustrate certain embodiments, and together with the written description, serve to explain certain principles of the microfluidic devices, systems, kits, computer readable media, methods, and related aspects disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 schematically shows an exemplary workflow for generating droplets, running one or more reactions (e.g., PCR, nucleic acid sequencing, etc.), and capturing the droplets in a microfluidic device for real-time analysis according to one exemplary embodiment.

FIG. 2 schematically shows microfluidic devices and related aspects according to one exemplary embodiment. (A) The high-density trapping device may incorporate up to five modules as needed. Each module consists of a glass slide, PDMS sieve layer, and PDMS chamber layer with access ports. (B) A top view of the pseudo-sieve geometry shows the tightly spaced posts. Two highways run through each third of the device. (C) Droplets sit in the chamber above the posts. (D) Droplets are loaded until the chamber is full. Excess oil may pass through the posts to the outlet. (E) Droplets are pressure loaded into the inlet, and self-assemble into a grid while loading. Droplets are trapped against the walls and immobilized. Since no droplets may escape, the device exhibits 100% loading efficiency. (F) The removal of excess oil removes wasted space between droplets, such that they may be packed in a high-density array for highly efficient loading.

FIG. 3 schematically shows droplets loaded on microfluidic devices according to exemplary embodiments. (A) Droplets loaded into one embodiment of the device (without highways) experience compression. Compression was most significant in the central lanes of the device. Snapshots are shown of the proximal and distal areas. (B) With highways to help spread the flow of droplets evenly, droplets remain uniform throughout another embodiment of the device and experience only minimal compression due to high-density packing.

FIG. 4 shows data from an analysis of methylation levels of a tumor suppressor gene, CDO1. (A) Droplets were generated with target copies of bisulfite-converted methylated CDO1 at occupancies (λ) spanning 4 order of magnitude, from 0.04% to 11%. After PCR, the droplets were loaded into the trapping device and their fluorescence imaged with a wide-field camera. Droplets were identified and counted in ImageJ. (B) The occupancy was calculated from the Poissonian occupancy based on the ratio of negative to total droplets and compared to the expected occupancy. The lower two and highest occupancies were repeated twice. A linear fit was applied to the log-log curve of expected vs. calculated over the serial dilution. The slope of 0.98 is within 2% of the ideal slope of 1, and a R² value of 0.99 indicates an excellent fit.

FIG. 5 show that 120 pL and 600 pL droplets were generated without a flow-focusing device and that 1000 pL droplets were generated by the QX200 ddPCR system. Each droplet size underwent the same PCR protocol in a 96-well plate, and were subsequently loaded into the trapping device for imaging. Fluorescent images were acquired with the same wide-field camera and macro lens and the same working distance. Bright-field images were all acquired under an Olympus microscope at 4× magnification. All systems were compatible with the range of droplet sizes.

FIG. 6 show that after loading, the device was sealed on both ends and placed on the thermal-optical system for real-time analysis. To demonstrate immobilization even at high, challenging temperatures, fluorescent images of the droplet capture region were acquired during thermal ramping from room temperature to 90° C., shown here in 5° intervals. A superimposed grid over a sub-region of the device pinpoints the position of each droplet from frame-to-frame. Droplets remained immobilized throughout in order to facilitate analysis of individual droplets.

FIG. 7 show real-time fluorescence monitoring data. (A) Fluorescent images of droplets containing amplicons of mixed epialleles were acquired during temperature ramping. Each epiallele denatures at distinct temperatures, measured as a loss of fluorescence of the dsDNA intercalating dye (Evagreen). (B) The average fluorescence of each droplet is plotted against temperature to obtain a melt curve. The negative derivation of the curve contains a peak, which is identified as the melt temperature (Tₘ) of the sequence. By identifying the Tₘ of each amplicon, a profile of the methylation heterogeneity within the sample may be obtained and analyzed.

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, cranial implant devices, and component parts, the following terminology, and grammatical variants thereof, will be used in accordance with the definitions set forth below.

About: As used herein, “about” or “approximately” as applied to one or more values or elements of interest, refers to a value or element that is similar to a stated reference value or element. In certain embodiments, the term “about” or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Detect: As used herein, “detect,” “detecting,” or “detection” refers to an act of determining the existence or presence of one or more characteristics, properties, states, or conditions in a subject, in a sample obtained or derived from a subject, or in a device, system, or component thereof.

Subject: As used herein, “subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.” For example, a subject can be an individual who has been diagnosed with having a cancer, is going to receive a cancer therapy, and/or has received at least one cancer therapy. The subject can be in remission of a cancer.

### DETAILED DESCRIPTION

This application discloses a microfluidic platform with 100% loading efficiency in some embodiments for high-density droplet trapping and real-time monitoring that is universally compatible with other droplet systems. In certain embodiments, the platform consists of a microfluidic droplet trapping device and a thermal-optical platform for parallelized real-time analyses across a wide range of temperatures. The trapping device typically employs a simple, passive immobilization strategy that is fundamentally compatible with any droplet size, and therefore is congruent with existing commercial droplet platforms.

Microfluidic droplet technologies have greatly enhanced the sensitivity and throughput of single-cell and single-molecule analyses. Discretization of samples into thousands or millions of digital droplets facilitates rapid detection of rare biomarkers and also enables new insights into cellular and molecular populations. Towards this end, commercial platforms like BioRad ddPCR™ and RainDance RainDrop™ are increasingly becoming turnkey tools for digital biomarker analysis. While most of these droplet platforms rely on sequential measurements of each droplet and its assay reactants at a single predetermined time point (“endpoint analysis”), many applications typically require the collection of time-resolved data (“real-time analysis). To achieve such generalizable real-time analysis of droplets, this application discloses a universally-compatible, fully-integrated platform that, in certain embodiments, consists of: (1): a microfluidic droplet imaging chamber that includes a pseudo-sieve to immobilize droplets with a packing density as high as 110,000 droplets per square inch; (2) a flatbed heater with controllable temperature ramping capability; and a (3) real-time wide-field optical imager. Together, the platform enables simultaneous real-time measurements of all droplets across a wide range of temperatures, fully leveraging the throughput of droplet microfluidics.

Traditional microfluidic droplet platforms invariably rely on sequential measurements of assay reactants in droplets at predetermined time points (“endpoint analysis”). These platforms therefore lack the capability to address a host of applications that involve time-resolved measurements for real-time analysis. Examples of such applications include genetic melting curve analysis, cell growth monitoring, and enzyme kinetics observations. The microfluidic devices, systems, and related aspects disclosed in this application facilitate increased utility of droplet microfluidics by enabling real-time analysis and molecular profiling of reactants in microfluidic droplets across a vast range of temperatures, while maintaining droplet stability. The microfluidic devices, systems, and related aspects disclosed in this application achieve increased generalizability by being universally-adaptable to essentially any existing droplet generation platform. Furthermore, the platform and related aspects disclosed herein fully leverages the throughput of droplet technologies by realizing simultaneous (as opposed to sequential) measurements of all droplets in a device, vastly reducing the time required for droplet analysis.

In certain embodiments, the platform or system disclosed herein consists of: (1) a microfluidic sieve that can immobilize droplets with at least about 110,000 droplets/in² packing density; (2) a flatbed heater with controllable temperature ramping capability; and a (3) real-time wide-field optical imager. Together, the platform enables simultaneous real-time measurements of all droplets across a wide range of temperatures.

Microfluidic Devices

Surface Tension Mediated Immobilization and Arrangement

In certain embodiments, the microfluidic device includes a microfluidic chamber, consisting of a tightly-spaced post array at its base, forms the basis of the device. Upon entering the chamber, droplets are under substantially constant compression from the low ceiling. Droplets realize a reduction in surface tension when sitting in the space between posts, which leads to temporary and controllable immobilization. The position and spacing of the posts facilitates a grid-like arrangement of droplets once fully loaded in the device.

Microfluidic Sieve for High-Density Immobilization

The microfluidic posts function as a pseudo-sieve, whereby droplets are restricted to the upper compartment above the posts while the carrier phase fluid (as well as any smaller debris) flow below the droplets, through the bottom around the posts and exit via the outlet of the device.

Universally Adaptable Droplet Immobilization

The pseudo-sieve pattern and surface-tension immobilization strategy applies to any aqueous droplet-based discretization. Therefore, the microfluidic devices disclosed herein can be easily adapted to any droplet platform capable of droplet generation, allowing for increased generalizability across a vast dynamic range.

Droplet Stability and Minimal Movement Across a Large Temperature Range

Droplets can be loaded into the device to maximum capacity, after which the inlets and outlets are temporarily blocked using a sealed syringe needle (or other such sealant) during analysis in some embodiments. Droplets are typically confined due to surface-tension immobilization and high-density packing, and therefore remain in place for the duration of real-time measurements.

Device Reusability

Flushing the device with carrier phase fluid from the outlet provides a means of fully clearing each device of all droplets and allowing for repeated use and potentially increased throughput of detection in some embodiments.

Off-Chip Droplet Processing and Multi-Module Functionality

For multistep assays that contain time-consuming reaction or incubation steps, droplets may be produced off-chip allowing high-throughput parallelization of time-limiting reaction steps on conventional 96- or 384-well plates. Droplets may then be immobilized in the devices disclosed herein for simultaneous assessment of multiple reactions, a range of reaction conditions, or large patient/sample cohorts. Multiple trapping units on a single device allow for high-throughput parallel analysis of distinct reactions in certain embodiments.

Thermal-Optical Measurement Platform

Simultaneous Measurements of all Droplets

In some embodiments, the systems or platforms disclosed herein contain wide-field imaging capabilities, fully leveraging the throughput of droplet platforms by realizing simultaneous (as opposed to sequential) measurements of all droplets in a device, vastly reducing the time required for real-time droplet analysis.

Real-Time Measurements of Droplets

In some embodiments, the thermal and optical imager work in tandem to record time-resolved data, demonstrated from millisecond through minute intervals, from droplets across a large range of temperatures, i.e. up to about 95° C., for effective real-time analysis.

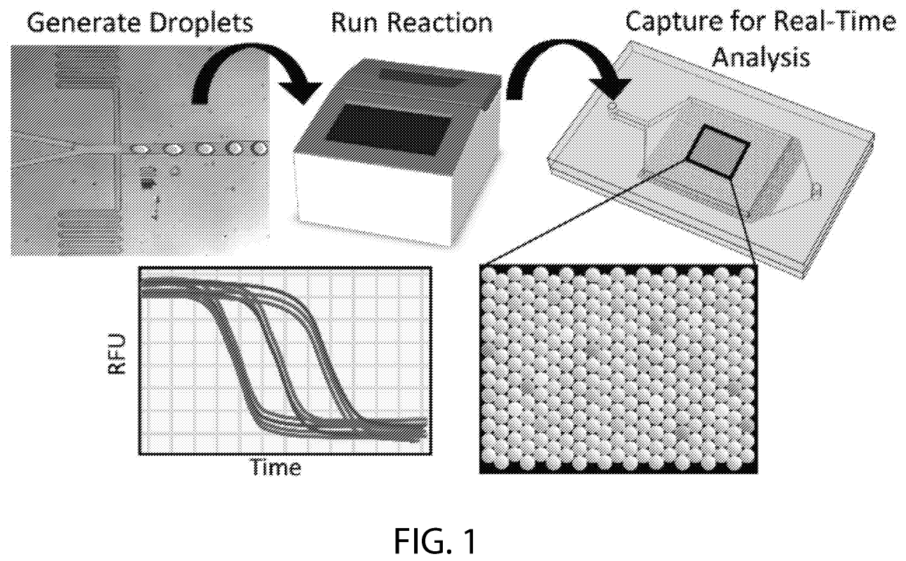

To further illustrate, FIG. 1 schematically depicts an exemplary workflow using the devices disclosed herein. As shown, following droplet formation, the droplets are loaded into the microfluidic imaging chamber by using a Tygon tubing adapter or the like (not within view). Optionally, droplets may be directly generated on the same device or a passive vacuum-assisted loading scheme is used. The microfluidic pseudo-sieve is typically fabricated from an elastomeric material (e.g., polydimethylsiloxane (PDMS) or the like), and the geometry and spacing of individual posts in the post array are fixed for optimal trapping. Optionally, other device material and geometry are used to also enhance trapping efficiency and droplet density. In some embodiments, fluorescence detection is performed in one color using a single illumination wavelength. In other embodiments, to expand the molecular profiling capability and throughput of the platform, multiple illumination sources and detection channels are used in a given application. In some embodiments, the application of the platforms disclosed herein is for genetic/epigenetic profiling using melting curve analysis. However, the platform can readily be used for other applications that use, for example, different assay and different temperature parameters. In some embodiments, aqueous-phase droplets are loaded and analyzed, but a solid-phase or slightly-compressible partition can also optionally be loaded at high-density and analyzed. In addition, digital confinement is typically used for single-molecule profiling, but can also be applied for multi-molecular monitoring (such as interactions, merging, etc.). Additional details related to microfluidic devices and systems that are optionally adapted for use with the devices and systems disclosed herein are also described in, for example, U.S. Patent Application Publication Nos. US 2019/0154715, US 2014/0106462, and US 2018/0304267, which are each incorporated by reference.

The term “computer system” is used herein to encompass any data processing system or processing unit or units. The computer system may include one or more processors or processing units. The computer system can also be a distributed computing system. The computer system may include, for example, a desktop computer, a laptop computer, a handheld computing device such as a PDA, a tablet, a smartphone, etc. A computer program product or products may be run on the computer system to accomplish the functions or operations described herein. The computer program product includes a computer readable medium or storage medium or media having instructions stored thereon used to program the computer system to perform the functions or operations described herein. Examples of suitable storage medium or media include any type of disk including floppy disks, optical disks, DVDs, CD ROMs, magnetic optical disks, RAMS, EPROMs, EEPROMs, magnetic or optical cards, hard disk, flash card (e.g., a USB flash card), PCMCIA memory card, smart card, or other media. Alternatively, a portion or the whole computer program product can be downloaded from a remote computer or server via a network such as the internet, an ATM network, a wide area network (WAN) or a local area network.

Stored on one or more of the computer readable media, the program may include software for controlling both the hardware of a general purpose or specialized computer system or processor. The software also enables the computer system or processor to interact with a user via output devices such as a graphical user interface, head mounted display (HMD), etc. The software may also include, but is not limited to, device drivers, operating systems and user applications. Alternatively, instead or in addition to implementing the methods described herein as computer program product(s) (e.g., as software products) embodied in a computer, the methods described herein can be implemented as hardware in which for example an application specific integrated circuit (ASIC) or graphics processing unit or units (GPU) can be designed to implement the method or methods, functions or operations of the present disclosure.

### Example

To demonstrate the utility of this platform, heterogeneous DNA methylation of a tumor suppressor gene, CDO1^{54,55}, was through digital PCR followed by digital High Resolution Melt (HRM). HRM is a facile post-PCR sequence fingerprinting technique that assesses sequence variations by observing sequence-specific release of DNA intercalating dye during thermal denaturation. HRM has been utilized in many applications, including bacterial identification^{30,56,82}, SNP detection^{57,58}, and biomarker analysis^{31,59,60}. HRM can greatly enhance sensitivity and analysis of rare methylated biomarkers in cell-free DNA^{31,32}, which has been investigated as an early biomarker for many cancers⁶¹⁻⁶³. This example shows how this platform can enhance the throughput of HRM-based analysis of rare molecules by performing droplet digital HRM (ddHRM) to discriminate 0%, 50%, and 100% methylated CDO1 on the device.

Experimental

Device Fabrication

Two photomasks were designed in L-Edit v16.0 (Tanner EDA) for the upper chamber and for the pseudo-sieve post array and were printed onto high-quality transparency (CAD/Art Services). To fabricate the molds, two silicon wafers were dehydrated for at least 6 hours, and oxygen-plasma treated at 85 W for 1 min (Technics PE-II). For the upper chamber, SU-8 3050 was spun at 3000 rpm for 30 s, baked at 65° C. for 1 min and 95° C. for 15 min, and exposed at 180 mJ/cm2. For the pseudo-sieve layer, the SU-8 3025 was spun at 1800 rpm for 30 s, baked at 65° C. for 1 min and 95° C. for 14 min, and then exposed at 175 mJ/cm². Following exposure, both wafers were baked for 1 min at 65° C. and 5 min at 95° C., developed with SU-8 developer, and baked at 200° C. for 1 hr.

The devices were fabricated from the master molds through soft lithography with 30 g of PDMS (Ellsworth), mixed at a ratio of 10:1 (w/w). The two layers were oxygen-plasma treated at 40 W for 45 s for bonding. A glass microscope slide (TedPella) and a coverglass slip were then oxygen-plasma treated at 40 W for 45 s and bonded to the bottom and top of the device, respectively. The devices were baked overnight at 80° C., and desiccated for 2 hours. Prior to use, FC-40 oil was vacuum-loaded into the device.

Droplet Generation and ddPCR

Dilute amounts of target DNA suspended in PCR reagents were digitized into tens of thousands of 100 pL, 600 pL, or 1 nL water-in-oil droplets. The aqueous phase consisted of DNA spiked into 1×QX200™ ddPCR™ EvaGreen® supermix (BioRad) and 3 μM primers (IDT). The oil phase used was QX200™ Droplet Generation Oil for EvaGreen (BioRad). For all 100 pL and 600 pL droplets, custom PDMS devices were designed with flow-focusing junctions spanning 60 μm and 84 μm respectively. The aqueous phase was first drawn into a 100-cm-long section of Tygon tubing (Cole-Parmer) with an inner diameter of 500 μm. The Tygon tubing section was then connected to a Hamilton 1000 glass syringe (Sigma-Aldrich) containing FC-40 oil (Sigma-Aldrich), which served as the displacement fluid for injecting the aqueous phase into the device using a syringe pump at a flow rate of 640 μL/h. Droplet generation oil was injected into the device using a separate syringe pump at 2400 μL/h. Generated droplets were collected from the device's outlet into a 1.5 mL DNA LoBind Tube (Eppendorf). 1 nL droplets were generated separately by assembling aqueous and oil phases into a commercially available DG8™ cartridge that was then inserted into a BioRad QX200™ Droplet Generator.

Droplets were transferred to a thermal cycler to initiate the PCR reaction. After carefully removing any excess oil, all generated droplets were carefully pipetted into a semi-skirted 96-well plate (BioRad) in 40 μL aliquots. The plate was heat-sealed with pierceable foil in the PX1 PCR plate sealer (Bio-Rad). The plate was then loaded into a C1000 Touch Thermal Cycler, and droplets underwent a thermocycling protocol of 5 min at 95° C. and 60 cycles of {30 s at 95° C., 30 s at 56° C., 30 s at 72° C.}, with 2° C./s ramp rate between each cycle.

Post-PCR droplets were finally loaded into the droplet array for real-time monitoring. First, droplets were carefully drawn into 30-cm-long sections of Tygon tubing by applying negative pressure using a syringe. One end of the Tygon tubing was then connected to a Hamilton 1000 syringe containing FC-40 displacement fluid. A 23 gauge needle was connected to the other end of the Tygon tubing to serve as the interface into the droplet array. A syringe pump was used to slowly flow the droplets into the droplet array first at 50 μL/h. As droplets stably entered the array, their flow rate was incrementally ramped up to 100 μL/h. Device loading was observed using a custom microscope with a 4× objective and a CCD camera. Loading continued until the droplets reached the outlet wall of the droplet array.

Real-Time Droplet Monitoring

The loaded device was sealed with epoxy-filled needles and placed on the thermal-optical platform. The device was illuminated by a 490 nm LED array (Thorlabs). A wide-field mirrorless interchangeable lens camera (Sony) acquired fluorescent images through a 526-LP emission filter (Omega). To simultaneously acquire high resolution melt curves from each droplet, a fluorescent image was acquired at 1 s intervals with 0.8 s exposure during thermal ramping on a flatbed heater (MJ Scientific) at a rate of 0.05° C./s.

Image Analysis

To perform droplet segmentation and identification, the first 10 images were loaded into ImageJ and averaged⁶⁴. The resulting image underwent morphological opening, followed by a morphological segmentation of radius gradient 1 to segment and identify each droplet⁶⁵. The droplet mask was then exported to Matlab for analysis along with the subsequent images. An automated script (Matlab) extracted the average raw fluorescence for each droplet at each temperature point and binned the values in 0.3° C. intervals to generate a melt curve for each droplet. After Savitzky-Golay filtering of each curve, the negative derivative was taken to identify the peak, or melt temperature (Tₘ) of the droplet amplicon. The methylation density of the amplicon in each droplet was identified by thresholding.

Results and Discussion

Overview of ddHRM

The platform or system presented in this example enables high-density trapping and immobilization of droplets for real-time monitoring and detection of rare molecules or variants. The platform consists of two main components: (1) the droplet trapping and immobilization device, which loads and immobilizes droplets at 100% efficiency by means of a sieve-like floor, and (2) the thermaloptical imaging platform, which acquires fluorescent images of all droplets within the device at specified temperatures. This platform was implemented to perform high resolution melt (HRM) analysis for detection and discrimination of rare methylated biomarkers.

A technique termed DREAMing, Discrimination of Rare EpiAlleles by Melt, was previously developed as a facile means of detecting various methylation patterns on a molecule-by-molecule basis amongst high background³². Briefly, the technique utilizes methylation-preferred or methylation-agnostic primers to amplify all bisulfite-converted methylation patterns of a given locus at single-copy sensitivity. Next, the sequences are discriminated by HRM by observing the sequence-dependent release of a DNA intercalating dye during thermal ramping. However, this technique performs quasi-digital analysis via conventional multiwell plates with limited dynamic range of detection and sensitivity, thereby undermining its practicality for clinical use. To address this, a microfluidic digital array platform called HYPER-Melt was developed in which this technique was implemented in a high density array of nanowells³¹. This microfluidic digital array platform enables detection and discrimination of methylated variants at frequencies as low as 0.00005%. The platform was further improved by increasing the loading efficiency from 12% to 80%33. However, this on-chip integrated system suffers from low throughput due to minimal parallelization of the time-consuming PCR step (three hours) required before dHRM analysis (five minutes).

Therefore, it was sought to implement this technique on a digital droplet platform in order to significantly improve performance in terms of sensitivity and throughput. Whereas the array-based device is limited to performing ˜4 to 6 PCR reactions in parallel on a single thermocycler, a commercial ddPCR system such as the BioRad QX200 may digitize as many as 8 samples into droplets at once, and can perform the time-limiting PCR step on as many as 96 samples simultaneously. By incorporating off-chip PCR thermocycling with a multi-module droplet trapping device and HRM platform, the system can realize both higher loading efficiency and much higher throughput.

To perform droplet digital HRM (ddHRM), the reaction mix was compartmentalized into droplets by flow-focusing. Droplets for each sample to be analyzed were loaded into a well of a 96-well plate and amplified by PCR in parallel on a thermal cycler. Next, droplets were loaded into the trapping device, and melt curves were acquired from each amplicon via the thermal-optical platform. After analysis, a population profile of various methylation levels was generated for each sample.

Device Architecture

100% Efficient Loading and Trapping Mechanism

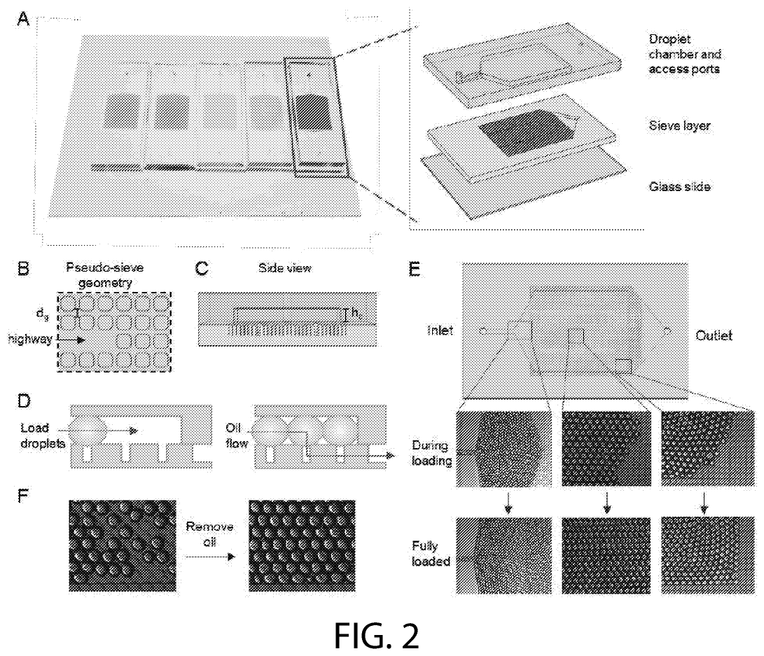

The high-density packing device consists of a glass microscope slide at the base and two PDMS layers, an upper droplet chamber and a lower pseudo-sieve floor (FIG. 2 (panel (A))). Up to five modules may be assembled in parallel onto a single glass slide as needed. Within each module, the pseudo-sieve layer is comprised of tightly spaced eight-sided polygonal posts (FIG. 2 (panel (B))). The intermittent top surfaces of each post are defined as the “floor” of the droplet chamber. The continuous surface at the bottom, between each post, is considered the “basement” of the device. A “highway”, or space without posts, runs between columns at approximately each third of the device to evenly space the droplets and relieve pressure.

The grid-like arrangement of the posts creates an array of gaps in the floor of diameter dg between the corners of each post. Upon entering the chamber, the height of the ceiling (hc) is less than the spherical height of each droplet, such that droplets are under slight compression between the ceiling and the floor (FIG. 2 (panel (C))). Droplets favor placement over the gaps due to the decrease in surface tension⁴⁷. The tight spacing between posts prevents droplets from escaping below the chamber into the basement of the device, whereas any excess oil between droplets may pass between the posts, through the basement, to the outlet of the device (FIG. 2 (panel (D))).

Droplets are pressure-loaded into the device inlet with a syringe pump (FIG. 2 (panel (E))). Upon reaching the droplet chamber, droplets self-assemble into a grid-like arrangement over the posts. Once reaching the end or side walls of the chamber, droplets are unable to escape, thereby demonstrating 100% efficiency in loading. Excess space occupied by oil in the droplet chamber can be removed through the basement to achieve 100% efficiency in trapping at a packing density of 110,000 droplets per in² (FIG. 2 (panel (F))).

Scalable Droplet Capacity

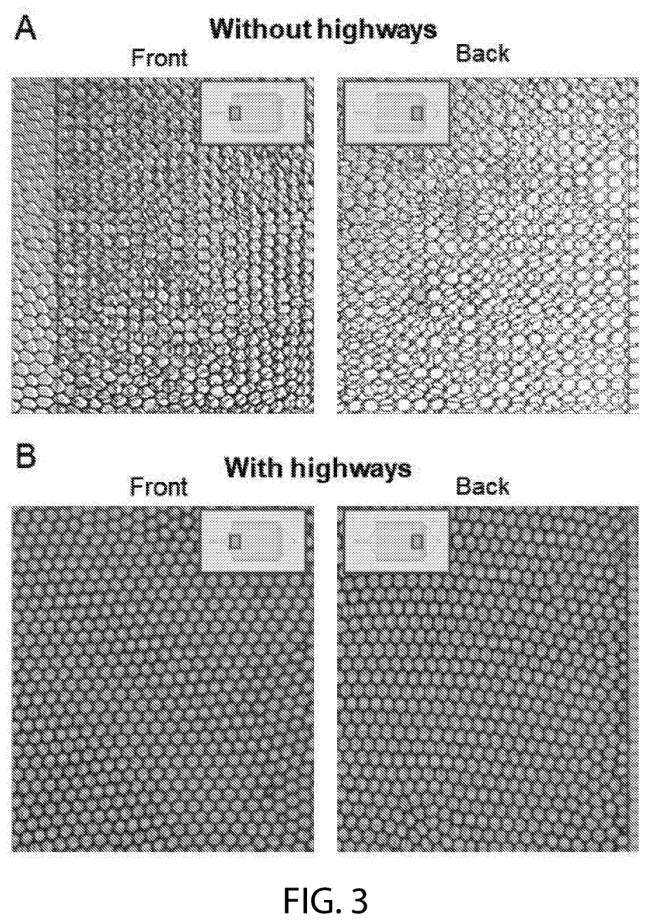

In most single-point disseminating loading mechanisms, wherein droplets are propelled by pressure-driven flow, a loaded while maintaining droplet stability³⁸. In earlier generations of the device design, it was observed increased compression of droplets due to pressure during filling of the final corners of the device, especially at the center regions on each end (FIG. 3 (panel (A))), which could compromise droplet integrity.

To address this, “highways” were implemented to reduce back-pressure and to facilitate even distribution of droplets throughout the device chamber during loading. The highways run through the initial 75% of the device, whereupon they cease to ensure trapping at the distal end. After implementing highways between each third of the device, droplets were observed to exhibit uniform, minimal compression throughout the device (FIG. 3 (panel (B))). Therefore, the device design is fundamentally scalable to higher droplet capacities.

Parallelized Real-Time Droplet Analysis

Detection and Quantification of Rare Molecules

Although a key goal in developing this device was to provide real-time monitoring capabilities, the device and imaging platform may also be utilized for highly accurate quantification and detection of rare molecules. While current commercial platforms may include separate fluorescent readout instrumentation, they provide no means of visual inspection and droplets are irrecoverable after analysis. Droplets in the high-density packing device may be quantified with any fluorescence platform as well as bright-field microscopy to allow for visual inspection of the droplets.

To validate the accuracy of the system, a serial dilution of synthetic DNA, representative of bisulfite-converted 100% methylated CDO1, was partitioned into 500 pL droplets, amplified in a 96-well plate, and then loaded onto the trapping device. The device was placed on the thermal-optical platform to acquire a single wide-field fluorescent image. The droplets were segmented in ImageJ, whereupon the number of positive droplets was calculated from the Poissonian occupancy equation.

The assay was also cross-validated by performing the same protocol in bulk and with the Biorad QX200 system.

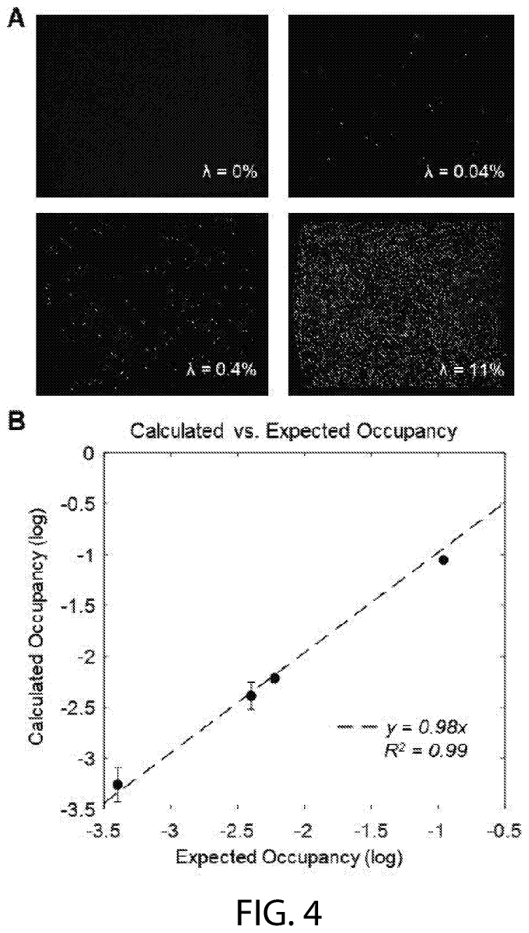

The expected occupancy of the synthetic target ranged from 11% to 0.04%. The calculated vs. expected occupancies are shown in FIG. 4 (log-log). The lower occupancies were repeated thrice while the higher occupancies were tested once. A linear regression curve was fit in Matlab, producing a slope of 0.98 with an R² of 0.99. These results demonstrate the utility of this platform for highly accurate droplet quantification, and provide a simple alternative to complex commercial droplet readers.

System Versatility

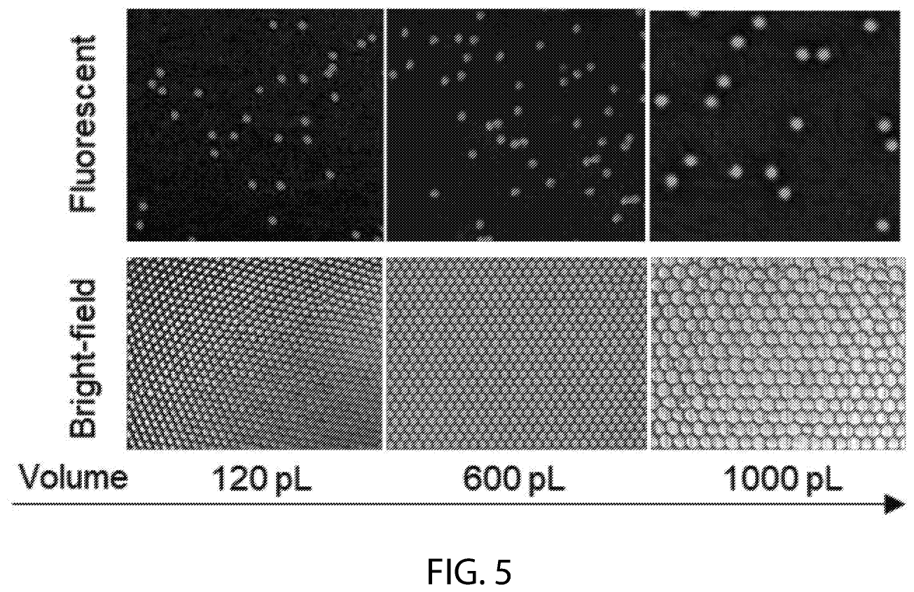

Due to the wide range of applications utilizing droplet technology, a fundamental aspect of the device design was versatility in droplet generation platforms and in droplet size. Therefore, the loading performance of the device was assessed across a range of common droplet volumes. Devices capable of producing 100 pL, 600 pL, and 1000 pL droplets using flow-focusing discretization were designed. The devices included geometric modifications of the flow-focusing nozzle used in devices we have previously developed⁶⁶⁻⁶⁸. Droplets were also generated using the BioRad QX200 platform and assessed for compatibility with the trapping device and real-time system. The device demonstrated 100% efficiency in loading of droplets ranging from 100 pL to 1 nL (FIG. 5).

The increasing number of applications of droplet technology may be attributed to the increased availability and reliability of commercial droplet platforms⁶⁹, such as BioRad's QX200 droplet system and RainDance's RainDrop Plus platform. In addition, increased research into surfactants and other droplet stabilizers^{3,51,70,71} has propelled the development of microfabricated droplet generation systems^{2-5,72,73}. With this device, it was demonstrated that loading and analysis of droplets generated by both the microfluidic droplet generation device as well as droplets generated by the Biorad QX200 system in this example. By integrating this device with other droplet systems, real-time analysis of droplets can be achieved without compromising throughput.

Droplet Immobilization and Real-Time Imaging

For time-lapse analyses of densely-packed systems, droplet immobilization is critical to reduce the computational burden of droplet identification and ensure analytical confidence. It was demonstrated that droplet immobilization on the device by evaluating the position of post-PCR droplets in time-lapse fluorescent images during thermal ramping. A mastermix was prepared with synthetic DNA representative of bisulfite-converted methylated CDO1, from which 600 pL droplets were generated in a flow-focusing device. After ddPCR in a 96-well plate, the droplets were loaded into the trapping device. The device was sealed on both ends and placed on the thermal-optical platform.

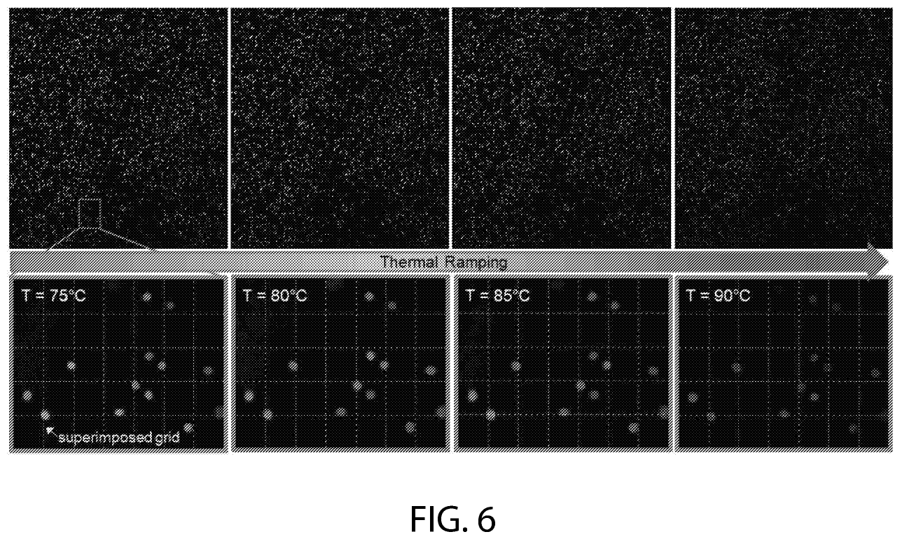

Fluorescent images were acquired of the droplet trapping chamber during thermal ramping (FIG. 6). A grid is superimposed over a sub-region of the device to illustrate the relative position of droplets between frames. Even at high temperatures, droplets remain immobilized and stable, validating the robustness of the trapping mechanism. This simple immobilization technique does not require any external equipment, and provides a facile means to maintain discretization of signals from adjacent droplets in densely-packed high-throughput systems.

Parallelized Droplet Digital High Resolution Melt (ddHRM)

This example illustrated the utility of this system or platform towards analysis of heterogeneous populations by performing droplet digital High Resolution Melt (ddHRM) to discriminate molecule-by-molecule methylation patterns of bisulfite-converted DNA. Synthetic DNA molecules representative of bisulfite-converted 0%, 50%, and 100% methylated CDO1 were digitized into 600 pL droplets. Droplets were then aliquoted into a 96-well plate and amplified in a thermal cycler following a standard PCR protocol with methylation-preferred primers as previously described³². Next, droplets were loaded onto the droplet trapping device and placed on the thermal-optical platform for HRM analysis.

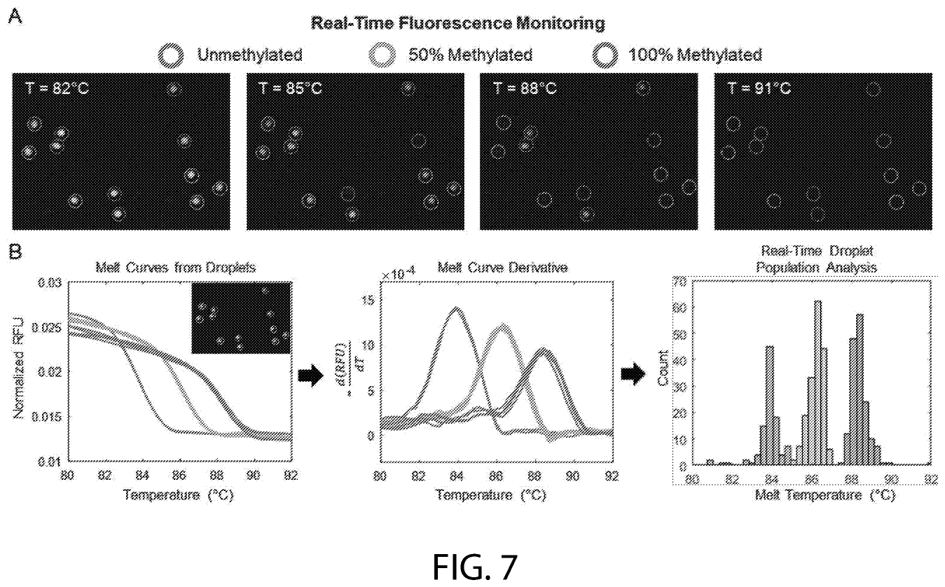

Wide-field fluorescent images of the droplet trapping region were acquired at 0.3° C. intervals during temperature ramping. To extract information from each individual droplet, the images were first segmented in ImageJ to identify the position of each droplet (FIG. 7 (panel (A))). Next, an automated Matlab script extracts the fluorescent information from each droplet at each temperature interval to generate melt curves (FIG. 7 (panel (B))). The peak of the negative derivative of each curve defines the melt temperature (Tₘ) of each sequence. A digital melt histogram then displays the Tₘ of each amplicon, which clearly depicts three distinct populations. The methylation density of each droplet can then be classified by its Tₘ, providing a quantitative population profile of methylation heterogeneity.

DNA methylation is one of the most-commonly studied epigenetic alterations in cancer ^{progression74-77}. Recent studies have shown that variable methylation levels within a locus correlate with disease progression^{78,79}. Furthermore, many models predict that methylation levels are highly variable early in carcinogenesis^{53,80,81}. This device and platform will enable, for example, further study into the effects of variable methylation on cancer etiology.

A high-density droplet trapping device and thermal-optical platform was developed for time-lapse analyses of up to 30,000 droplets in parallel in this example. Single molecules of DNA were compartmentalized, amplified, and quantified at high accuracy across three orders of magnitude at concentrations as low as 0.8 copies per μL. The utility of this platform was demonstrated by profiling variable methylation levels of a tumor suppressor gene (CDO1) with ddHRM.

The pseudo-sieve functionality of the droplet chamber enables 100% loading and trapping efficiency, thus the device is highly suitable for analysis of rare molecules or variants. The simple, passive immobilization strategy is compatible with droplets of different sizes and many readily-available imaging modalities. The effective mitigation of backpressure was demonstrated, indicating that the design is readily-scalable to higher droplet capacities and throughput.

To further illustrate, optionally, droplets can be recovered by reversing the direction of flow in the device in some embodiments. In addition, in certain embodiments, the throughput is further increased in both sample number and droplet capacity. For example, within a chamber, although the scaling potential is not expected to be limited by back-pressure, a larger droplet chamber with a high aspect ratio (w/h) may experience sagging during fabrication. This can be simply addressed by incorporating, for example, support posts throughout the larger device. In other embodiments, higher resolution photolithography techniques are utilized to produce finer spacing in the pseudo-sieve layer. This typically permits even smaller droplets to be captured and analyzed in the trapping region. Incorporating this embodiment also typically leads to increased dynamic range and sensitivity of the platform. Among other attributes, these techniques will help to development a better understanding of population heterogeneity and improve detection of rare biomarkers, among other features.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, kits, devices, and/or component parts or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

### REFERENCES

(1) Kaushik, A. M.; Hsieh, K.; Wang, T.-H. Droplet Microfluidics for High-Sensitivity and High-Throughput Detection and Screening of Disease Biomarkers. Wiley Interdiscip. Rev. Nanomedicine Nanobiotechnology 2018, e1522.
(2) Joensson, H. N.; Andersson Svahn, H. Droplet Microfluidics—A Tool for Single-Cell Analysis. Angewandte Chemie—International Edition. 2012, pp 12176-12192.
(3) Theberge, A. B.; Courtois, F.; Schaerli, Y.; Fischlechner, M.; Abell, C.; Hollfelder, F.; Huck, W. T. S. Microdroplets in Microfluidics: An Evolving Platform for Discoveries in Chemistry and Biology. Angew. Chemie—Int. Ed. 2010, 49 (34), 5846-5868.
(4) Ding, Y.; Choo, J.; DeMello, A. J. From Single-Molecule Detection to next-Generation Sequencing: Microfluidic Droplets for High-Throughput Nucleic Acid Analysis. Microfluid. Nanofluidics 2017, 21 (3), 58.
(5) Murphy, T. W.; Zhang, Q.; Naler, L. B.; Ma, S.; Lu, C. Recent Advances in the Use of Microfluidic Technologies for Single Cell Analysis. Analyst. 2018, pp 60-80.
(6) Choi, K.; Ng, A. H. C.; Fobel, R.; Wheeler, A. R. Digital Microfluidics. Annu. Rev. Anal. Chem. is online Annu. Rev. Anal. Chem 2012, 5, 413-440.
(7) Pekin, D.; Skhiri, Y.; Baret, J.-C.; Corre, D. Le; Mazutis, L.; Salem, C. Ben; Millot, F.; Harrak, A. El; Hutchison, J. B.; Larson, J. W.; et al. Quantitative and Sensitive Detection of Rare Mutations Using Droplet-Based Microfluidics. Lab Chip 2011, 11 (13), 2156-2166.
(8) Yelleswarapu, V.; Buser, J. R.; Haber, M.; Baron, J.; Inapuri, E.; Issadore, D. Mobile Platform for Rapid Sub-Picogramper-Milliliter, Multiplexed, Digital Droplet Detection of Proteins. Proc. Natl. Acad. Sci. 2019, 116 (10), 4489-4495.
(9) Shuga, J.; Zeng, Y.; Novak, R.; Lan, Q.; Tang, X.; Rothman, N.; Vermeulen, R.; Li, L.; Hubbard, A.; Zhang, L.; et al. Single Molecule Quantitation and Sequencing of Rare Translocations Using Microfluidic Nested Digital PCR. Nucleic Acids Res. 2013, 41 (16), 1-11.
(10) Zec, H.; Shin, D. J.; Wang, T.-H. Novel Droplet Platforms for the Detection of Disease Biomarkers. Expert Rev. Mol. Diagn. 2014, 14 (7), 787-801.
(11) Teh, S.-Y.; Lin, R.; Hung, L.-H.; Lee, A. P. Droplet Microfluidics.
(12) Boettger, L. M.; Handsaker, R. E.; Zody, M. C.; McCarroll, S. A. Structural Haplotypes and Recent Evolution of the Human 17q21.31 Region. Nat. Genet. 2012, 44 (8), 881-885.
(13) Miotke, L.; Lau, B. T.; Rumma, R. T.; Ji, H. P. High Sensitivity Detection and Quantitation of DNA Copy Number and Single Nucleotide Variants with Single Color Droplet Digital PCR. Anal. Chem. 2014, 86 (5), 2618-2624.
(14) Beck, J.; Hennecke, S.; Bornemann-Kolatzki, K.; Urnovitz, H. B.; Neumann, S.; Ströbel, P.; Kaup, F. J.; Brenig, B.; Schütz, E. Genome Aberrations in Canine Mammary Carcinomas and Their Detection in Cell-Free Plasma DNA. PLoS One 2013, 8 (9), e75485.
(15) Beaver, J. A.; Jelovac, D.; Balukrishna, S.; Cochran, R. L.; Croessmann, S.; Zabransky, D. J.; Wong, H. Y.; Valda Toro, P.; Cidado, J.; Blair, B. G.; et al. Detection of Cancer DNA in Plasma of Patients with Early-Stage Breast Cancer. Clin. Cancer Res. 2014, 20 (10), 2643-2650.
(16) Zhu, G.; Ye, X.; Dong, Z.; Lu, Y. C.; Sun, Y.; Liu, Y.; McCormack, R.; Gu, Y.; Liu, X. Highly Sensitive Droplet Digital PCR Method for Detection of EGFR-Activating Mutations in Plasma Cell-Free DNA from Patients with Advanced Non-Small Cell Lung Cancer. J. Mol. Diagnostics 2015, 17 (3), 265-272.
(17) Goh, H.-G.; Lin, M.; Fukushima, T.; Saglio, G.; Kim, D.; Choi, S.-Y.; Kim, S.-H.; Lee, J.; Lee, Y.-S.; Oh, S.-M.; et al. Sensitive Quantitation of Minimal Residual Disease in Chronic Myeloid Leukemia Using Nanofluidic Digital Polymerase Chain Reaction Assay. Leuk. Lymphoma 2011, 52 (5), 896-904.
(18) Jackson, J. B.; Choi, D. S.; Luketich, J. D.; Pennathur, A.; Ståhlberg, A.; Godfrey, T. E. Multiplex Preamplification of Serum DNA to Facilitate Reliable Detection of Extremely Rare Cancer Mutations in Circulating DNA by Digital PCR. J. Mol. Diagn. 2016, 18 (2), 235-243.
(19) Mitchell, P. S.; Parkin, R. K.; Kroh, E. M.; Fritz, B. R.; Wyman, S. K.; Pogosova-Agadjanyan, E. L.; Peterson, A.; Noteboom, J.; O'Briant, K. C.; Allen, A.; et al. Circulating MicroRNAs as Stable Blood-Based Markers for Cancer Detection. Proc. Natl. Acad. Sci. 2008, 105 (30), 10513-10518.
(20) McDermott, G. P.; Do, D.; Litterst, C. M.; Maar, D.; Hindson, C. M.; Steenblock, E. R.; Legler, T. C.; Jouvenot, Y.; Marrs, S. H.; Bemis, A.; et al. Multiplexed Target Detection Using DNA Binding Dye Chemistry in Droplet Digital PCR. Anal. Chem. 2013, 85 (23), 11619-11627.
(21) Gilg, I. C.; Archer, S. D.; Floge, S. A.; Fields, D. M.; Vermont, A. I.; Leavitt, A. H.; Wilson, W. H.; Martinez, J. Differential Gene Expression Is Tied to Photo Chemical Efficiency Reduction in Virally Infected Emiliania Huxleyi. Mar. Ecol. Prog. Ser. 2016, 555, 13-27.
(22) Tadmor, A. D.; Ottesen, E. A.; Leadbetter, J. R.; Phillips, R. Probing Individual Environmental Bacteria for Viruses by Using Microfluidic Digital PCR. Science (80-.). 2011, 333 (6038), 58-62.
(23) Pholwat, S.; Stroup, S.; Foongladda, S.; Houpt, E. Digital PCR to Detect and Quantify Heteroresistance in Drug Resistant Mycobacterium tuberculosis. PLoS One 2013, 8 (2), e57238.
(24) Schuler, F.; Trotter, M.; Geltman, M.; Schwemmer, F.; Wadle, S.; Dominguez-Garrdo, E.; López, M.; Cervera-Acedo, C.; Santibáñez, P.; Von Stetten, F.; et al. Digital Droplet PCR on Disk. Lab Chip 2016, 16 (1), 208-216.
(25) Leng, X.; Yang, C. J. Agarose Droplet Microfluidics for Highly Parallel and Efficient Single Molecule Emulsion PCR. Methods Mol. Biol. 2013, 949 (21), 413-422.
(26) Hindson, B. J.; Ness, K. D.; Masquelier, D. A.; Belgrader, P.; Heredia, N. J.; Makarewicz, A. J.; Bright, I. J.; Lucero, M. Y.; Hiddessen, A. L.; Legler, T. C.; et al. High-Throughput Droplet Digital PCR System for Absolute Quantitation of DNA Copy Number. Anal. Chem. 2011, 83 (22), 8604-8610.
(27) Chen, J.; Luo, Z.; Li, L.; He, J.; Li, L.; Zhu, J.; Wu, P.; He, L. Capillary-Based Integrated Digital PCR in Picoliter Droplets. Lab Chip 2018, 18 (3), 412-421.
(28) Fraley, S. I.; Hardick, J.; Jo Masek, B.; Athamanolap, P.; Rothman, R. E.; Gaydos, C. A.; Carroll, K. C.; Wakefield, T.; Wang, T.-H.; Yang, S.; et al. Universal Digital High-Resolution Melt: A Novel Approach to Broad-Based Profiling of Heterogeneous Biological Samples. Nucleic Acids Res. 2013, 41 (18), e175-e175.
(29) Andini, N.; Wang, B.; Athamanolap, P.; Hardick, J.; Masek, B. J.; Thair, S.; Hu, A.; Avornu, G.; Peterson, S.; Cogill, S.; et al. Microbial Typing by Machine Learned DNA Melt Signatures. Sci. Rep. 2017, 7, 42097.
(30) Athamanolap, P.; Parekh, V.; Fraley, S. I.; Agarwal, V.; Shin, D. J.; Jacobs, M. A.; Wang, T.-H.; Yang, S. Trainable High Resolution Melt Curve Machine Learning Classifier for Large-Scale Reliable Genotyping of Sequence Variants. PLoS One 2014, 9 (10), e109094.
(31) O'Keefe, C. M.; Pisanic, T. R.; Zec, H.; Overman, M. J.; Herman, J. G.; Wang, T. Facile Profiling of Molecular Heterogeneity by Microfluidic Digital Melt. Sci. Adv. 2018, 4 (9), eaat6459.
(32) Pisanic, T. R.; Athamanolap, P.; Poh, W.; Chen, C.; Hulbert, A.; Brock, M. V; Herman, J. G.; Wang, T.-H.; Wang, T.-H. DREAMing: A Simple and Ultrasensitive Method for Assessing Intratumor Epigenetic Heterogeneity Directly from Liquid Biopsies. Nucleic Acids Res. 2015, 43 (22), e154.
(33) O'Keefe, C. M.; Giammanco, D.; Li, S.; Pisanic, T. R.; Wang, T. H. J. Multilayer Microfluidic Array for Highly Efficient Sample Loading and Digital Melt Analysis of DNA Methylation. Lab Chip 2019.
(34) Selck, D. A.; Ismagilov, R. F. Instrument for Real-Time Digital Nucleic Acid Amplification on Custom Microfluidic Devices. PLoS One 2016, 11 (10).
(35) Jackson-Holmes, E. L.; McDevitt, T. C.; Lu, H. A Microfluidic Trap Array for Longitudinal Monitoring and Multi-Modal Phenotypic Analysis of Individual Stem Cell Aggregates. Lab Chip 2017, 17 (21), 3634-3642.
(36) Hansen, C. L.; Lecault, V.; Vanlnsberghe, M.; Sekulovic, S.; Knapp, D. J. H. F.; Wohrer, S.; Bowden, W.; Viel, F.; McLaughlin, T.; Jarandehei, A.; et al. High-Throughput Analysis of Single Hematopoietic Stem Cell Proliferation in Microfluidic Cell Culture Arrays. Nat. Methods 2011.
(37) Sjostrom, S. L.; Joensson, H. N.; Svahn, H. A. Multiplex Analysis of Enzyme Kinetics and Inhibition by Droplet Microfluidics Using Picoinjectors 3. 2013, 13, 1754.
(38) Schmitz, C. H. J.; Rowat, A. C.; Köster, S.; Weitz, D. A. Dropspots: A Picoliter Array in a Microfluidic Device. Lab Chip 2009, 9 (1), 44-49.
(39) Labanieh, L.; Nguyen, T. N.; Zhao, W.; Kang, D. K. Floating Droplet Array: An Ultrahigh-Throughput Device for Droplet Trapping, Real-Time Analysis and Recovery. Micromachines 2015, 6 (10), 1469-1482.
(40) Terekhov, S. S.; Smirnov, I. V; Stepanova, A. V; Bobik, T. V; Mokrushina, Y. A.; Ponomarenko, N. A.; Belogurov, A. A.; Rubtsova, M. P.; Kartseva, O. V; Gomzikova, M. O.; et al. Microfluidic Droplet Platform for Ultrahigh-Throughput Single-Cell Screening of Biodiversity. Proc. Natl. Acad. Sci. 2017, 114 (10), 2550-2555.
(41) Hua, Z.; Rouse, J. L.; Eckhardt, A. E.; Srinivasan, V.; Vamsee, K.; Schell, W. A.; Benton, J. L.; Mitchell, T. G.; Michael, G.; Carolina, N. Multiplexed Real-Time Polymerase Chain Reaction on a Digital Microfluidic Platform. Anal. Chem 2010, 82 (6), 2310-2316.
(42) Selck, D. A.; Ismagilov, R. F. Instrument for Real-Time Digital Nucleic Acid Amplification on Custom Microfluidic Devices. PLoS One 2016, 11 (10), 1-20.
(43) Boukellal, H.; Selimović, E.; Jia, Y.; Cristobal, G.; Fraden, S. Simple, Robust Storage of Drops and Fluids in a Microfluidic Device. Lab Chip 2009, 9 (2), 331-338.
(44) Hatch, A. C.; Fisher, J. S.; Tovar, A. R.; Hsieh, A. T.; Lin, R.; Pentoney, S. L.; Yang, D. L.; Lee, A. P. 1-Million Droplet Array with Wide-Field Fluorescence Imaging for Digital PCR. Lab Chip 2011, 11 (22), 3838.
(45) Bian, X.; Jing, F.; Li, G.; Fan, X.; Jia, C.; Zhou, H.; Jin, Q.; Zhao, J. A Microfluidic Droplet Digital PCR for Simultaneous Detection of Pathogenic Escherichia coli O157 and Listeria monocytogenes. Biosens. Bioelectron. 2015, 74, 770-777.
(46) Kim, S. H.; Iwai, S.; Araki, S.; Sakakihara, S.; Iino, R.; Noji, H. Large-Scale Femtoliter Droplet Array for Digital Counting of Single Biomolecules. Lab Chip 2012, 12 (23), 4986-4991.
(47) Abbyad, P.; Dangla, R.; Alexandrou, A.; Baroud, C. N. Rails and Anchors: Guiding and Trapping Droplet Microreactors in Two Dimensions. Lab Chip 2011, 11 (5), 813-821.
(48) Huebner, A.; Bratton, D.; Whyte, G.; Yang, M.; DeMello, A. J.; Abell, C.; Hollfelder, F. Static Microdroplet Arrays: A Microfluidic Device for Droplet Trapping, Incubation and Release for Enzymatic and Cell-Based Assays. Lab Chip 2009, 9 (5), 692-698.
(49) Balaban, N. Q.; Merrin, J.; Chait, R.; Kowalik, L.; Leibler, S. Bacterial Persistence as a Phenotypic Switch. Science (80-.). 2004, 305 (5690), 1622-1625.
(50) Safa, N.; Vaithiyanathan, M.; Sombolestani, S.; Charles, S.; Melvin, A. T. Population-Based Analysis of Cell-Penetrating Peptide Uptake Using a Microfluidic Droplet Trapping Array. Anal. Bioanal. Chem. 2019.
(51) Courtois, F.; Olguin, L. F.; Whyte, G.; Bratton, D.; Huck, W. T. S.; Abell, C.; Hollfelder, F. An Integrated Device for Monitoring Time-Dependent in Vitro Expression from Single Genes in Picolitre Droplets. ChemBioChem 2008, 9 (3), 439-446.
(52) Athamanolap, P.; Hsieh, K.; Chen, L.; Yang, S.; Wang, T.-H. Integrated Bacterial Identification and Antimicrobial Susceptibility Testing Using PCR and High-Resolution Melt. Anal. Chem 2017, 89, 33.
(53) Pisanic, T. R.; Athamanolap, P.; Wang, T. H. Defining, Distinguishing and Detecting the Contribution of Heterogeneous Methylation to Cancer Heterogeneity. Seminars in Cell and Developmental Biology. 2017, pp 5-17.
(54) Harada, H.; Hosoda, K.; Moriya, H.; Mieno, H.; Ema, A.; Ushiku, H.; Washio, M.; Nishizawa, N.; Ishii, S.; Yokota, K.; et al. Cancer-Specific Promoter DNA Methylation of Cysteine Dioxygenase Type 1 (CDO1) Gene as an Important Prognostic Biomarker of Gastric Cancer. PLoS One 2019, 14 (4), e0214872.
(55) Brait, M.; Ling, S.; Nagpal, J. K.; Chang, X.; Park, H. L.; Lee, J.; Okamura, J.; Yamashita, K.; Sidransky, D.; Kim, M. S. Cysteine Dioxygenase 1 Is a Tumor Suppressor Gene Silenced by Promoter Methylation in Multiple Human Cancers. PLoS One 2012, 7 (9), e44951.
(56) Abou Tayoun, A. N.; Burchard, P. R.; Caliendo, A. M.; Scherer, A.; Tsongalis, G. J. A Multiplex PCR Assay for the Simultaneous Detection of Chlamydia trachomatis, Neisseria gonorrhoeae, and Trichomonas vaginalis. Exp. Mol. Pathol. 2015, 98 (2), 214-218.
(57) Reed, G. H.; Wittwer, C. T. Sensitivity and Specificity of Single-Nucleotide Polymorphism Scanning by High-Resolution Melting Analysis. Clin. Chem. 2004, 50 (10), 1748-1754.
(58) Gonzalez-Bosquet, J.; Calcei, J.; Wei, J. S.; Garcia-Closas, M.; Sherman, M. E.; Hewitt, S.; Vockley, J.; Lissowska, J.; Yang, H. P.; Khan, J.; et al. Detection of Somatic Mutations by High-Resolution DNA Melting (HRM) Analysis in Multiple Cancers. PLoS One 2011, 6 (1), e14522.
(59) Hanson, E. K.; Ballantyne, J. Rapid and Inexpensive Body Fluid Identification by RNA Profiling-Based Multiplex High Resolution Melt (HRM) Analysis. F1000Research 2014, 2, 281.
(60) Yang, X.; Dai, W.; Kwong, D. L. W.; Szeto, C. Y. Y.; Wong, E. H. W.; Ng, W. T.; Lee, A. W. M.; Ngan, R. K. C.; Yau, C. C.; Tung, S. Y.; et al. Epigenetic Markers for Noninvasive Early Detection of Nasopharyngeal Carcinoma by Methylation-Sensitive High Resolution Melting. Int. J. Cancer 2015, 136 (4), E127-E135.
(61) Schwarzenbach, H.; Hoon, D. S. B.; Pantel, K. Cell-Free Nucleic Acids as Biomarkers in Cancer Patients. Nat. Rev. Cancer 2011, 11 (6), 426-437.
(62) Mikeska, T.; Bock, C.; Do, H.; Dobrovic, A. DNA Methylation Biomarkers in Cancer Progress towards Clinical Implementation. Expert Rev. Mol. Diagn. 2012, 12 (5), 473-487.
(63) Yokoi, K.; Yamashita, K.; Watanabe, M. Analysis of DNA Methylation Status in Bodily Fluids for Early Detection of Cancer. Int. J. Mol. Sci. 2017, Vol. 18, Page 735 2017, 18 (4), 735.
(64) Abràmoff, M. D.; Magalhães, P. J.; Ram, S. J. Image Processing with ImageJ. Biophotonics International. 2004.
(65) Legland, D.; Arganda-Carreras, I.; Andrey, P. MorphoLibJ: Integrated Library and Plugins for Mathematical Morphology with ImageJ. Bioinformatics 2016.
(66) Mach, K. E.; Kaushik, A. M.; Hsieh, K.; Wong, P. K.; Wang, T. H.; Liao, J. C. Optimizing Peptide Nucleic Acid Probes for Hybridization-Based Detection and Identification of Bacterial Pathogens. Analyst 2019.
(67) Guan, W.; Chen, L.; Rane, T. D.; Wang, T. H. Droplet Digital Enzyme-Linked Oligonucleotide Hybridization Assay for Absolute RNA Quantification. Sci. Rep. 2015.
(68) Kaushik, A. M.; Hsieh, K.; Chen, L.; Shin, D. J.; Liao, J. C.; Wang, T. H. Accelerating Bacterial Growth Detection and Antimicrobial Susceptibility Assessment in Integrated Picoliter Droplet Platform. Biosens. Bioelectron. 2017.
(69) Baker, M. Digital PCR Hits Its Stride. Nat. Methods 2012, 9 (6), 541-544.
(70) Yin, K.; Zeng, X.; Liu, W.; Xue, Y.; Li, X.; Wang, W.; Song, Y.; Zhu, Z.; Yang, C. J. Stable Colloidosomes Formed by Self-Assembly of Colloidal Surfactant for Highly Robust Digital PCR. Anal. Chem. 2019, acs.analchem.9b00470.
(71) Fu, Y.; Zhou, H.; Jia, C.; Jing, F.; Jin, Q.; Zhao, J.; Li, G. A Microfluidic Chip Based on Surfactant-Doped Polydimethylsiloxane (PDMS) in a Sandwich Configuration for Low-Cost and Robust Digital PCR. Sensors Actuators B Chem. 2017, 245, 414-422.
(72) Du, G.; Fang, Q.; den Toonder, J. M. J. Microfluidics for Cell-Based High Throughput Screening Platforms—A Review. Analytica Chimica Acta. January 2016, pp 36-50.
(73) Pompano, R. R.; Liu, W.; Du, W.; Ismagilov, R. F. Microfluidics Using Spatially Defined Arrays of Droplets in One, Two, and Three Dimensions. Annu. Rev. Anal. Chem. (Palo Alto. Calif.). 2011, 4, 59-81.
(74) Jones, P. a; Baylin, S. B. The Fundamental Role of Epigenetic Events in Cancer. Nat. Rev. Genet. 2002, 3 (6), 415-428.
(75) Herman, J. G.; Baylin, S. B. Gene Silencing in Cancer in Association with Promoter Hypermethylation. n engl j med 2003, 34921349, 2042-2054.
(76) Dawson, M. A.; Kouzarides, T. Cancer Epigenetics: From Mechanism to Therapy. Cell 2012, 150, 12-27.
(77) Sandoval, J.; Esteller, M. Cancer Epigenomics: Beyond Genomics. Current Opinion in Genetics and Development. 2012, pp 50-55.
(78) Li, S.; Garrett-Bakelman, F.; Chung, S.; Sanders, M.; Hricik, T.; Rapaport, F.; Mason, C. Distinct Evolution and Dynamics of Epigenetic and Genetic Heterogeneity in Acute Myeloid Leukemia. Nat. Med. 2016, 22, 792-799.
(79) Sheffield, N. C.; Pierron, G.; Klughammer, J.; Datlinger, P.; Schönegger, A.; Schuster, M.; Hadler, J.; Surdez, D.; Guillemot, D.; Lapouble, E.; et al. DNA Methylation Heterogeneity Defines a Disease Spectrum in Ewing Sarcoma. Nat. Med. 2017, 23 (3), 386-395.
(80) Timp, W.; Feinberg, A. P. Cancer as a Dysregulated Epigenome Allowing Cellular Growth Advantage at the Expense of the Host. Nat. Rev. Cancer 2013, 13 (7), 497-510.
(81) Teschendorff, A. E.; Widschwendter, M. Differential Variability Improves the Identification of Cancer Risk Markers in DNA Methylation Studies Profiling Precursor Cancer Lesions. Bioinformatics 2012, 28 (11), 1487-1494.
(82) Athamanolap, P; Hsieh K.; O'Keefe C. M.; Zhang Y.; Yang S.; and Wang T-H, Machine Learning-Assisted Digital PCR and Melt Enables Broad Bacteria Identification and Pheno-Molecular Antimicrobial Susceptibility Test. Cold Spring Harbor Laboratory 2019 preprint.

1. A microfluidic device, comprising a body structure having a droplet compression chamber that is structured to at least partially and simultaneously contain a plurality of droplets, at least one sieve structure in fluid communication with the droplet compression chamber, which sieve structure comprises an array of protrusions that extend from at least one surface of the body structure and define at least a portion of one or more fluidic circuits, and at least one port at least partially disposed in the body structure, which port is in fluid communication with the droplet compression chamber and/or the fluidic circuits, wherein the droplet compression chamber and sieve structure compress and selectively immobilize the plurality of droplets and permit selective removal of carrier fluid from the droplet compression chamber through the fluidic circuits, when the plurality of droplets are disposed in the droplet chamber and positioned substantially above the fluidic circuits proximal to the array of protrusions.

2. The microfluidic device of claim 1, wherein the body structure comprises a first layer defining at least the portion of the droplet compression chamber that is structured to at least partially and simultaneously contain the plurality of droplets, and a second layer operably connected to the first layer, which second layer comprises the at least one sieve structure in fluid communication with the droplet compression chamber, wherein the at least one port is at least partially disposed in the first and/or second layer.

3. The microfluidic device of claim 1, comprising one or more gaps within and/or proximal to the array of protrusions, which gaps substantially lack protrusions.

4. The microfluidic device of claim 1, comprising the plurality of droplets.

5. The microfluidic device of claim 4, wherein the plurality of droplets comprises a partitioned sample.

6. The microfluidic device of claim 5, wherein the sample comprises one or more cells and/or biomolecules.

7. The microfluidic device of claim 1, wherein at least a portion of one or more of the protrusions comprise at least one cross-sectional shape selected from the group consisting of: a square, a rectangle, an oval, a trapezoid, a circle, an irregular n-sided polygon, and a regular n-sided polygon.

8. The microfluidic device of claim 1, wherein the droplet compression chamber is structured to at least partially and simultaneously contain the plurality of droplets at a density of at least about 110,000 droplets per square inch of at least one surface of the droplet compression chamber.

9. The microfluidic device of claim 1, comprising at least two ports at least partially disposed in the body structure and in fluid communication with the droplet compression chamber and/or the fluidic circuits, wherein at least a first port is configured to flow droplets into the droplet compression chamber and at least a second port is configured to flow carrier fluid out of the droplet compression chamber through the fluidic circuits.

10. A kit comprising the microfluidic device of claim 1.

11. A method of analyzing a sample, the method comprising:
receiving a mixture comprising a plurality of droplets and at least one carrier fluid in a droplet compression chamber of a microfluidic device through at least a first port of the microfluidic device that is in fluid communication with the droplet compression chamber, wherein the plurality of droplets comprises partitioned portions of the sample and wherein at least one sieve structure of the microfluidic device is in fluid communication with the droplet compression chamber, which sieve structure comprises an array of protrusions that extend from at least one surface of the microfluidic device and define at least a portion of one or more fluidic circuits;
removing at least a portion of the carrier fluid from the droplet compression chamber through at least a second port of the microfluidic device that is in fluid communication with the fluidic circuits to immobilize the plurality of droplets substantially above the fluidic circuits proximal to the array of protrusions to generate an immobilized population of droplets; and,
detecting at least one detectable signal from the immobilized population of droplets, thereby analyzing the sample.

12. The method of claim 11, wherein the plurality of droplets is in an aqueous phase and wherein the carrier fluid is in a non-aqueous phase.

13. The method of claim 11, wherein the detectable signal comprises a thermal and/or electromagnetic property of, or originating from, one or more members of the population of droplets or components thereof.

14. The method of claim 11, wherein the sample comprises one or more cells and/or biomolecules.

15. The method of claim 11, comprising detecting the detectable signal using a thermal and/or optical imaging device.

16. The method of claim 11, comprising at least about 110,000 droplets per square inch of at least one surface of the droplet compression chamber.

17. The method of claim 11, further comprising obtaining the sample from a subject and/or generating the plurality of droplets using at least one droplet generating device.

18. (canceled)

19. A microfluidic system, comprising:
a microfluidic device, comprising a body structure having a droplet compression chamber that is structured to at least partially and simultaneously contain a plurality of droplets, at least one sieve structure in fluid communication with the droplet compression chamber, which sieve structure comprises an array of protrusions that extend from at least one surface of the body structure and define at least a portion of one or more fluidic circuits, and at least one port at least partially disposed in the body structure, which port is in fluid communication with the droplet compression chamber and/or the fluidic circuits, wherein the droplet compression chamber and sieve structure compress and selectively immobilize the plurality of droplets and permit selective removal of carrier fluid from the droplet compression chamber through the fluidic circuits, when the plurality of droplets are disposed in the droplet chamber and positioned substantially above the fluidic circuits proximal to the array of protrusions;
a detection device configured to obtain detectable signal from the plurality of droplets, when the plurality of droplets are disposed in the droplet chamber and positioned substantially above the fluidic circuits proximal to the array of protrusions; and,
a control device operably connected to the detection device, which control device comprises, or is capable of accessing, computer readable media comprising non-transitory computer-executable instructions which, when executed by at least one electronic processor perform at least:
detecting at least one detectable signal from the plurality of droplets, when the plurality of droplets are disposed in the droplet chamber and positioned substantially above the fluidic circuits proximal to the array of protrusions.

20. The microfluidic system of claim 19, further comprising a droplet generating device operably connected to the control device, which control device further comprises, or is capable of further accessing, computer readable media comprising non-transitory computer-executable instructions which, when executed by the at least one electronic processor further perform:
generating the plurality of droplets using the droplet generating device.

21. The microfluidic system of claim 19, further comprising a droplet treatment device operably connected at least to the control device, the droplet generating device, and the microfluidic device, which control device further comprises, or is capable of further accessing, computer readable media comprising non-transitory computer-executable instructions which, when executed by the at least one electronic processor further perform:
conveying the plurality of droplets from the droplet generating device to the droplet treatment device;
treating the plurality of droplets received from the droplet generating device to generate treated droplets using the droplet treatment device; and,
conveying the treated droplets from the droplet treatment device to the microfluidic device.

22. (canceled)
